(19) United States
(12) Plant Patent Application Publication
(10) Pub. No.: US 2004/0187181 P1
(43) Pub. Date: Sep. 23, 2004

(54) Rose plant named 'WELzero'

(76) Inventor: Verlie Whitson Wells, Brighton, TN (US)

Correspondence Address: Verlie W. Wells, 471 Lucy Kelly Rd., Brighton, TN, 38011, US

(21) Appl. No.: 10/389,572

(22) Filed: Mar. 17, 2003

(51) Int. Cl. A01H 5/00

(52) U.S. Cl. PLT/139

(57) ABSTRACT

The present invention relates to a new and distinct variety of hardy, bush type rose plant of the hybrid tea class primarily identified by buds and flowers of a medium red color, exhibition form and semi-glossy green foliage.

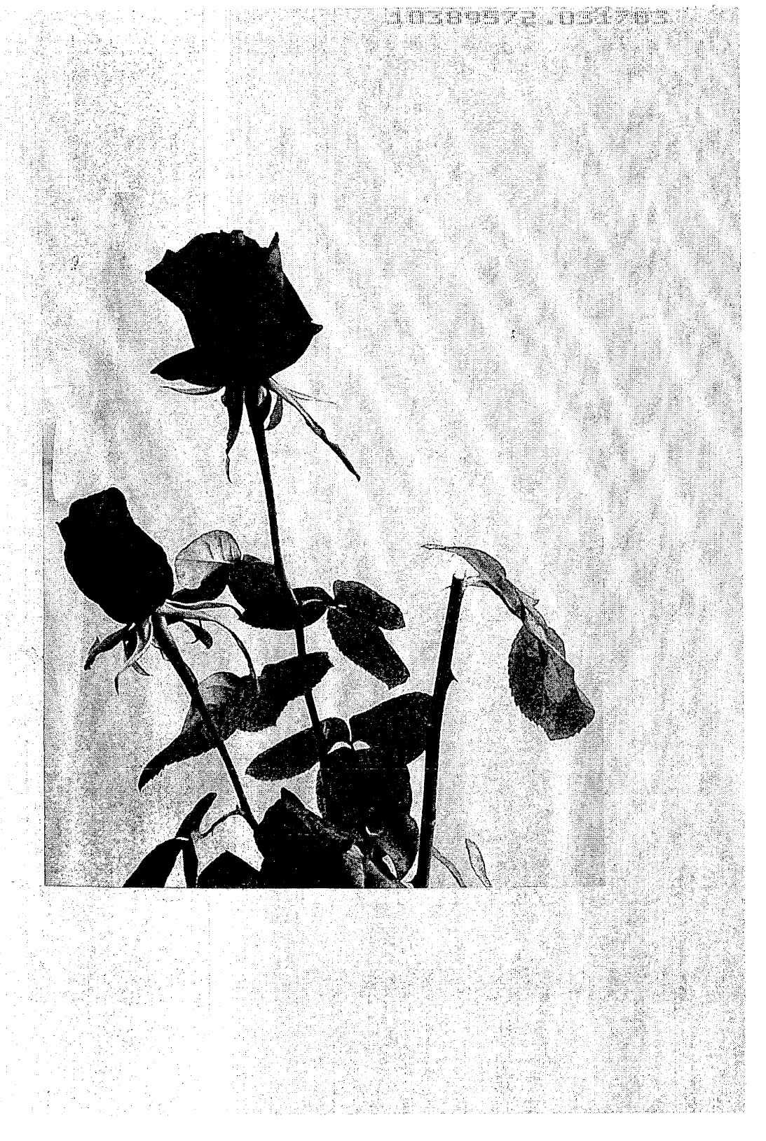

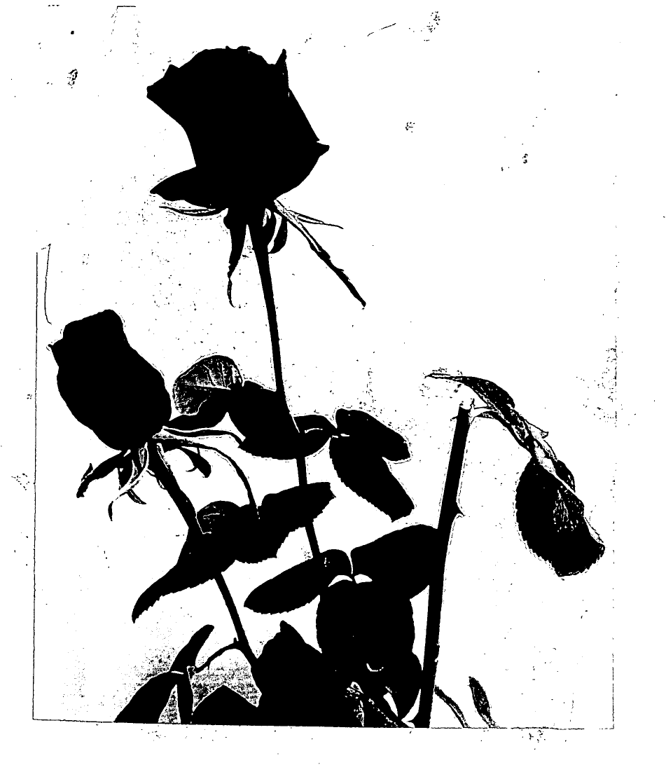

Be if known that I, Verlie W. Wells, a citizen of the United States of America, residing at Brighton, in the county of Tipton, State of Tennessee, have invented certain new and useful improvements in:

### ROSE PLANT/Var. WELzero

and I do declare the following to be a full, clear and exact description of the same.

The present invention relates to a new and distinct variety of hardy, bush type rose plant of the hybrid tea class; the variety being primarily characterized—as to novelty—by buds and flowers of a medium red color which generally are of exhibition form.

The variety is further characterized by:

An abundance of bloom, with flowers often borne singly and also several to the stem.

A plant which is vigorous, bushy, and upright in shape with medium growth habit and leathery/semi-glossy foliage.

The plant, which has an average height of 48 inches, can easily be maintained at a lesser height by occasional trimming.

A plant which grows and blooms satisfactorily both in the greenhouse and outdoors; can be grown in pots; and provides excellent garden decoration as well as cut flowers.

The present variety of hybrid tea rose was originated by me in my greenhouse located at Brighton, Tenn. under conditions of careful control and observation, as a cross between ‘Alec's Red’ as the seed parent and a seedling of ‘First Prize’ as the pollen parent.

Subsequent to origination of the variety, I successfully asexually reproduced it at my hybridizing garden, located as aforesaid, by cuttings; the reproductions having run true in all respects.

The color photograph illustrates the variety, including the foliage, buds and flowers.

Referring now specifically to the new and distinct variety of hybrid tea rose plant, the following is a detailed description thereof in outline; all major color plate identifications being by reference to The Royal Horticultural Society Colour Chart, except where common terms of color definition are employed.

Class: Hybrid Tea. Variety identification: WELzero (Commercial synonym: Nine-Eleven).

### FLOWER

Observations made from specimens grown in a garden environment at Brighton, Tenn. in April 2002 through October 2002.

Blooming habit: Repeats well.

Bud:

Size.—1 inch at base. 1¼ inches in height.

Form.—Urn shape.

Color.—Medium Red. Red Purple Group 60D.

Sepals.—Color: Medium to Dark Yellow Green. Inner Surface Yellow Green Group 145C. Outer Surface Yellow Green Group 147A. Surface texture: Mostly smooth. Number: Usually five with some foliaceous appendages.

Peduncle.—Length: 4 to 4½ inches. Surface: Small thorns. Strength: Stiff and strong. Upright. Color: Yellow Green Group 144B with streaks of Grey Red Group 182A.

Bloom:

Size.—Large. Average open size 4 to 4½ inches.

Borne.—Singly and in clusters of three flowers.

Stems.—Long and straight.

Form.—High centered.

Petalage.—Double; Number of petals under normal conditions: 25-35.

Color.—Medium Red. Upper side of petal Red Purple Group 60B. Lower side of petal Red Purple Group 60D.

Variegations.—None.

Discoloration.—May darken with age.

Fragrance.—None.

Reproductive parts:

Anthers.—Size: Medium. Color: Yellow Orange Group 23A. Arrangement: Regular around styles.

Filaments.—Yellow Orange Group 23A.

Pollen.—Quantity: Average.

Styles.—Color: Orange Group 29B.

Stigmas.—Color: Orange Group 29B.

Ovaries.—All enclosed in calyx.

Hips.—None. Female reproductive system appears to be sterile.

Seeds.—Has not demonstrated the ability to produce seeds.

Growth:

Habit.—Tall and upright, bushy, vigorous to 48 inches.

Foliage:

Leaflets.—Number on normal mid-stem: 5 to 7 leaves.

Size.—Medium. Leaflets vary in length from 1½ to 3½ inches.

Quantity.—Abundant.

Color.—New foliage: Upper Surface Green Group 143B. Lower Surface Green Group 137A; reddish overcast and reddish serrated edge. Old foliage: Upper surface: Green Group 137C. Lower surface: Green Group 143C.

Leaflets:

Shape.—Pointed oval.

Texture.—Leathery; semi-glossy.

Edge.—Serrated.

Serration.—Small to medium.

Petiole rachis.—Smooth with Grey Orange Group 177A coloration.

Petiole underside.—Smooth with Grey Orange Group 174A coloration.

Stipules.—Medium about ½ inch.

Wood:

New wood.—Color: Reddish but turns medium green very quickly.

Old wood.—Color: Medium Green Group 137B. Smooth bark.

Prickles:

Quantity.—Medium about ¾ of the way up the stem, then very few.

Form.—Varying sizes from small prickles to medium thorns; Medium thorn size (3/16 to ¼ inch); Prickles hook down from stem.

Color.—Grey Purple Group 184C.

The hybrid tea rose plant and its flowers as herein described may vary in slight detail due to climatic, soil and cultural conditions under which the variety may be grown; the present description being of the variety as grown at Brighton, Tenn.

The following is claimed:

1. A new and distinct variety of hybrid tea rose plant of hardy, upright habit, substantially as illustrated and described, characterized by buds and flowers of exhibition form, essentially medium red in color, with reverse of petals being light red in color, and further characterized by a plant of urn shape, vigorous, with the main stems and shoots being moderately thorny, the said plant being easy to propagate from cuttings, with an abundance of flowers borne singly or several per stem.
